(19) United States
(12) Patent Application Publication
(10) Pub. No.: US 2006/0024360 A1
(43) Pub. Date: Feb. 2, 2006

(54) Stable injectable composition of alpha tocopheryl succinate, analogues and salts thereof

(75) Inventor: Andrew Xian Chen, San Diego, CA (US)

Correspondence Address: SEED INTELLECTUAL PROPERTY LAW GROUP PLLC, 701 FIFTH AVE, SUITE 6300, SEATTLE, WA, 98104-7092, US

(73) Assignee: SD Pharmaceuticals, Inc., Carlsbad, CA (US)

(21) Appl. No.: 11/192,439

(22) Filed: Jul. 28, 2005

Related U.S. Application Data
(60) Provisional application No. 60/592,097, filed on Jul. 28, 2004.

(51) Int. Cl. A61K 31/337; A61K 9/127; A61K 31/355

(52) U.S. Cl. 424/450; 514/458; 514/449

(57) ABSTRACT

The present invention provides compositions that comprise alpha-tocopheryl succinate or its analogue or salt and methods for preparing and using such compositions.

### CROSS-REFERENCE TO RELATED APPLICATION

This application claims the benefit of U.S. Provisional Patent Application No. 60/592,097, filed Jul. 28, 2004, which is incorporated by reference in its entirety.

### BACKGROUND OF THE INVENTION

1. Field of the Invention

This invention relates to the field of compositions of alpha-tocopheryl succinate, analogues or salts thereof. In particular, this invention provides colloidal dispersion compositions of alpha-tocopheryl succinate, analogues or salts thereof that are stable, safe and efficacious.

2. Description of the Related Art

The greatest challenge in anticancer therapy lies with selectivity of the therapeutic agent; an effective anticancer drug must be highly selective for malignant cells while being free of deleterious efforts on normal cells. Unfortunately, to date, most anticancer drugs, especially in the family of chemotherapy agents, are known to possess high toxicity resulting in undesired side effects in patients and sadly such side effects are often so severe that they are either intolerable by the patient, causing deterioration of general health conditions of the patients, or negatively affect quality of life of the patient. The development of more selective and non-toxic therapeutic agents has become a new trend in the pursuit of new anticancer treatments.

Great hope has been given to micronutrients as anticancer agents, since they represent natural compounds with beneficial effects for normal cells and tissues. One of these is alpha-tocopheryl succinate (TS). Alpha-tocopheryl succinate, also known as vitamin E succinate, a semisynthetic vitamin E analogue, has been reported to have potent anticancer activities. Compared to the traditional chemotherapeutic agent, alpha-tocopheryl succinate is regarded as non-toxic (Zondlo Fiume, Int J Toxicol. 2002; 21 Suppl 3:51-116) and is metabolized to vitamin E, thereby yielding a compound with a secondary beneficial activity. Thus, alpha-tocopheryl succinate epitomizes a group of novel compounds that hold substantial promise as future anticancer drugs.

Alpha-tocopheryl succinate is a potent anticancer agent with a unique structure and pharmacokinetics in vivo. Alpha-tocopheryl succinate is highly selective for malignant cells, inducing them into apoptotic death largely via the mitochondrial route (Neuzil, Br J Cancer. Nov. 17, 2003; 89(10):1822-6).

Many researchers have reported the anticancer activities of alpha-tocopheryl succinate against various tumors based on in vivo or in vitro studies. Malafa et al. reported that alpha-tocopheryl succinate inhibited melanoma growth in mice (Surgery. January 2002; 131(1):85-91). Barnett demonstrated the activity of alpha-tocopheryl succinate in inhibiting color cancer liver metastases (J Surg Res. August 2002; 106(2):292-8). In immunocompromised mice, Tomassetti has shown that alpha-tocopheryl succinate suppressed malignant mesothelioma (Int J Cancer. May 1, 2004; 109(5):641-2). Liu et al. reported alpha-tocopheryl succinate inhibits human gastric carcinoma cell growth (Wei Sheng Yan Jiu. May 30, 2000; 29(3):172-4). All of these researchers seem to point out that alpha-tocopheryl succinate is a potent anticancer agent against various cancer cells with high selectivity.

The anticancer mechanism of alpha-tocopheryl succinate has also been well studied. Most researchers suggest that alpha-tocopheryl succinate induces apoptosis in cancer cells. Neuzil et al. published several papers on the study of the apoptotic mechanism of alpha-tocopheryl succinate (FASEB J. 15(2): 403-15, 2001; Redox Rep. 2001; 6(3):143-51; Biochem J. Mar. 15, 2002; 362(Pt 3):709-15).

The structure-and-apoptogenic activity relationship of alpha-tocopheryl succinate has also been well studied. The apoptogenic activity of alpha-tocopheryl succinate was found to be unique to the alpha-tocopheryl succinate structure and is not related to vitamin E. Kogure et al. reported that the terminal dicarboxylic moiety is required for the apoptotic activity of alpha-tocopheryl succinate (Biochim Biophys Acta. May 3, 2004; 1672(2):93-9). Amongst the analogues tested, the esters of alpha-tocopherol with dicarboxylic acids such as alpha-tocopheryl oxalate, alpha-tocopheryl malonate, along with alpha-tocopheryl succinate, were found to induce apoptosis in mouse cancer line (C1271), whereas the other tocopheryl analogs or esters tested including alpha-tocopheryl pimelate, alpha-tocopheryl succinate ethyl ester, alpha-tocopherol, gamma-tocopherol, alpha-tocopheryl nicotinate and alpha-tocopheryl acetate, were not apoptogenic. Alpha-tocopheryl oxalate was the most potent alpha-tocopheryl derivative tested.

Birringer et al. (Br J Cancer. Jun. 16, 2003; 88(12):1948-55) reported a significant difference in apoptogenic activity amongst alpha-tocopheryl succinate analogues. Analogues of alpha-tocopheryl succinate with lower numbers of methyl substitutions on the aromatic ring were less active than alpha-tocopheryl succinate. Replacement of the succinyl group with a maleyl group greatly enhanced the activity, while replacement of the succinyl group with a glutaryl group reduced the activity. Methylation of the free succinyl carboxyl group on alpha-tocopheryl succinate and delta- tocopheryl succinate completely eliminated the apoptogenic activity of the parent compounds. Alpha-tocotrienol (alpha-T3 H) failed to induce apoptosis, while gamma-T3 H was apoptogenic, and more so when succinylated. Shortening the aliphatic side chain of gamma-T3 by one isoprenyl unit increased its activity. Neither phytyl nor oleyl succinate caused apoptosis.

Other interesting biological findings of alpha-tocopheryl succinate include alpha-tocopheryl succinate' ability to enhance radiation-induced chromosomal damage levels in human cancer cells, but reduces the damage levels in normal cells (Kumar et al., J Am Coll Nutr. August 2002; 21(4):339-43), and to sensitize established tumors to vaccination with nonmatured dendritic cells (Ramanathapuram et al., Cancer Immunol Immunother. 2004; 53(7):580-8). The safety assessment of alpha-tocopheryl succinate has also been well documented (Zondlo Fiume, Int J Toxicol. 2002; 21 Suppl 3:51-116).

In most studies, the in vivo anticancer activity of alpha-tocopheryl succinate was demonstrated by intraperitoneal injection (i.p.) of alpha-tocopheryl succinate, which is dissolved in DMSO. Intraperitoneal (i.p.) administration of a therapeutic agent dissolved in DMSO is not a generally accepted procedure for humans. Alpha-tocopheryl succinate loses its anticancer activity if it is given orally since the succinate ester is cleaved in the gastrointestinal tract, yielding the parent alpha tocopherol, which lacks the apoptogenic activity.

Therefore for human therapeutic purposes, an intravenously injectable formulation (i.v.) of alpha-tocopheryl succinate is desired. To date, there appear to be only two alpha-tocopheryl succinate intravenous formulations. Kogure et al. reported a vesiculated alpha-tocopheryl succinate formulation that was administered to mice intravenously (Cancer Lett. Mar. 20, 2003; 192(1):19-24). This vesiculated alpha-tocopheryl succinate formulation contains alpha-tocopheryl succinate in phosphate buffered saline with pH adjusted to neutral with sodium hydroxide. The vesiculated alpha-tocopheryl succinate formulation was prepared by sonication and was characterized as a suspension with an average diameter of 350 nm.

Another intravenously injectable formulation of alpha-tocopheryl succinate (Jizomoto et al., Biochim Biophys Acta. Aug. 4, 1994; 1213(3):343-8) is a liposomal formulation that contains phosphatidylethanolamine and cholesterol. This formulation was designed as a pH-sensitive drug delivery vehicle capable of incorporating a drug at a neutral pH and releasing the drug in an acidic environment (i.e., cytosol).

Both the vesiculated and the liposomal formulations of alpha-tocopheryl succinate were found to be unstable by this inventor. When freshly prepared the formulations were white and milky. Over 2 week storage at 5° C. in dark in a sealed glass vials, these pH neutral and aseptically prepared formulations turned into yellow-green color with noticeable curd-like precipitates formed. An i.v. injection of such formulations into mice immediately caused death. It is believed that there have been extensive degradations of alpha-tocopheryl succinate (oxidation) and aggregation of the vesicles and liposomes. Hydrolysis of alpha-tocopheryl succinate in these formulations during the storage might also have occurred.

To develop a therapeutically feasible alpha-tocopheryl succinate product for human use, it is therefore desired to have a stable and intravenously injectable formulation for alpha-tocopheryl succinate that is free from any deleterious ingredients such as DMSO. Furthermore, it is likely that the lack of such a clinically feasible formulation has impeded the clinical development of alpha-tocopheryl succinate as an anti-cancer drug. The present invention meets the above needs and provides additional related advantages.

### BRIEF SUMMARY OF THE INVENTION

The present invention, in one aspect, provides new compositions of alpha-tocopheryl succinate that are stable and suitable for injection. In particular, this invention is directed to a composition that comprises alpha-tocopheryl succinate, an analogue or a salt thereof; at least one component selected from the group consisting of ah oil component, phospholipid, and antioxidant; and water; wherein the composition is a colloidal dispersion having an average particle size less than about 1000 nm (e.g., less than 200 nm) in diameter, and wherein the alpha-tocopheryl succinate, analogue or salt thereof is stable for at least 1 month (e.g., at least 6 months) at room temperature.

In certain embodiments, the present invention provides a pharmaceutical composition that comprises alpha-tocopheryl succinate, an analogue or a salt thereof, an oil component, and water, wherein the composition is a colloidal dispersion having an average particle size less than about 1000 nm (e.g., less than 200 nm) in diameter, and wherein the alpha-tocopheryl succinate, analogues or salts thereof is stable for at least 1 month (e.g., at least 6 months) at room temperature.

In certain embodiments, the present invention provides a pharmaceutical composition that comprises alpha-tocopheryl succinate, an analogue or a salt thereof, phospholipid, and water, wherein the composition is a colloidal dispersion having an average particle size less than about 1000 nm (e.g., less than 200 nm) in diameter, and wherein the alpha-tocopheryl succinate, analogues or salts thereof is stable for at least 1 month (e.g., at least 6 months) at room temperature.

In certain embodiments, the present invention provides a pharmaceutical composition that comprises alpha-tocopheryl succinate, an analogue or a salt thereof, an antioxidant, and water, wherein the composition is a colloidal dispersion having an average particle size less than about 1000 nm (e.g., less than 200 nm) in diameter, and wherein the alpha-tocopheryl succinate, analogues or salts thereof is stable for at least 1 month (e.g., at least 6 months) at room temperature.

In certain embodiments, the present invention provides a pharmaceutical composition that comprises alpha-tocopheryl succinate, an analogue or a salt thereof, an oil component, phospholipid, and water, wherein the composition is a colloidal dispersion having an average particle size less than about 1000 nm (e.g., less than 200 nm) in diameter, and wherein the alpha-tocopheryl succinate, analogues or salts thereof is stable for at least 1 month (e.g., at least 6 months) at room temperature.

In certain embodiments, the present invention provides a pharmaceutical composition that comprises alpha-tocopheryl succinate, an analogue or a salt thereof, an oil component, an antioxidant, and water, wherein the composition is a colloidal dispersion having an average particle size less than about 1000 nm (e.g., less than 200 nm) in diameter, and wherein the alpha-tocopheryl succinate, analogues or salts thereof is stable for at least 1 month (e.g., at least 6 months) at room temperature.

In certain embodiments, the present invention provides a pharmaceutical composition that comprises alpha-tocopheryl succinate, an analogue or a salt thereof, phospholipid, an antioxidant, and water, wherein the composition is a colloidal dispersion having an average particle size less than about 1000 nm (e.g., less than 200 nm) in diameter, and wherein the alpha-tocopheryl succinate, analogues or salts thereof is stable for at least 1 month (e.g., at least 6 months) at room temperature.

In certain embodiments, the present invention provides a pharmaceutical composition that comprises alpha-tocopheryl succinate, an analogue or a salt thereof, an oil component, phospholipid, an antioxidant, and water, wherein the composition is a colloidal dispersion having an average particle size less than about 1000 nm (e.g., less than 200 nm) in diameter, and wherein the alpha-tocopheryl succinate, analogues or salts thereof is stable for at least 1 month (e.g., at least 6 months) at room temperature.

In another aspect, the present invention provides a pharmaceutical composition that comprises alpha-tocopheryl succinate, an analogue or a salt thereof; at least one component selected from the group consisting of an oil component, phospholipid, and an antioxidant; and a cryoprotectant; wherein the composition is a dry solid, the alpha-tocopheryl succinate, analogues or salts thereof in the composition is stable for at least 1 month (e.g., at lest 6 months) at room temperature, and the dry solid, upon addition of water, forms a colloidal dispersion having an average particle size less than 1000 nm (e.g., less than 200 nm) in diameter.

In certain embodiments, the present invention provides a pharmaceutical composition that comprises alpha-tocopheryl succinate, an analogue or a salt thereof, an oil component, and a cryoprotectant; wherein the composition is a dry solid, the alpha-tocopheryl succinate, analogues or salts thereof in the composition is stable for at least 1 month (e.g., at lest 6 months) at room temperature, and the dry solid, upon addition of water, forms a colloidal dispersion having an average particle size less than 1000 nm (e.g., less than 200 nm) in diameter.

In certain embodiments, the present invention provides a pharmaceutical composition that comprises alpha-tocopheryl succinate, an analogue or a salt thereof, phospholipid, and a cryoprotectant; wherein the composition is a dry solid, the alpha-tocopheryl succinate, analogues or salts thereof in the composition is stable for at least 1 month (e.g., at lest 6 months) at room temperature, and the dry solid, upon addition of water, forms a colloidal dispersion having an average particle size less than 1000 nm (e.g., less than 200 nm) in diameter.

In certain embodiments, the present invention provides a pharmaceutical composition that comprises alpha-tocopheryl succinate, an analogue or a salt thereof, an antioxidant, and a cryoprotectant; wherein the composition is a dry solid, the alpha-tocopheryl succinate, analogues or salts thereof in the composition is stable for at least 1 month (e.g., at lest 6 months) at room temperature, and the dry solid, upon addition of water, forms a colloidal dispersion having an average particle size less than 1000 nm (e.g., less than 200 nm) in diameter.

In certain embodiments, the present invention provides a pharmaceutical composition that comprises alpha-tocopheryl succinate, an analogue or a salt thereof, an oil component, phospholipid, and a cryoprotectant; wherein the composition is a dry solid, the alpha-tocopheryl succinate, analogues or salts thereof in the composition is stable for at least 1 month (e.g., at lest 6 months) at room temperature, and the dry solid, upon addition of water, forms a colloidal dispersion having an average particle size less than 1000 nm (e.g., less than 200 nm) in diameter.

In certain embodiments, the present invention provides a pharmaceutical composition that comprises alpha-tocopheryl succinate, an analogue or a salt thereof, phospholipid, an antioxidant, and a cryoprotectant; wherein the composition is a dry solid, the alpha-tocopheryl succinate, analogues or salts thereof in the composition is stable for at least 1 month (e.g., at lest 6 months) at room temperature, and the dry solid, upon addition of water, forms a colloidal dispersion having an average particle size less than 1000 nm (e.g., less than 200 nm) in diameter.

In certain embodiments, the present invention provides a pharmaceutical composition that comprises alpha-tocopheryl succinate, an analogue or a salt thereof, an oil component, phospholipid, an antioxidant, and a cryoprotectant; wherein the composition is a dry solid, the alpha-tocopheryl succinate, analogues or salts thereof in the composition is stable for at least 1 month (e.g., at lest 6 months) at room temperature, and the dry solid, upon addition of water, forms a colloidal dispersion having an average particle size less than 1000 nm (e.g., less than 200 nm) in diameter.

In certain embodiments, the pharmaceutical compositions are chemically stabilized by the addition of stabilizers and/or removal of water. For example, the stabilizers may be antioxidants, and the removal of water may be accomplished by freeze-drying, vacuum drying, or spray drying.

In certain embodiments, the colloidal dispersion of this invention is physically stabilized by addition of an oil component, phospholipid and optionally a cryoprotectant, wherein the colloidal dispersion is a submicron-sized suspension, or oil-in-water emulsion.

In certain embodiments, the pharmaceutical compositions of the invention are chemically stable for at least 1, 2, 3, 4, 5, or 6 months at room temperature, wherein the loss of intact alpha-tocopheryl succinate (or its analogue or salt) is no more than about 15% by at least 1, 2, 3, 4, 5, or 6 months. In certain embodiments, the loss of intact alpha-tocopheryl succinate (or its analogue or salt) is no more than about 10%, 7.5%, or 5%.

In certain embodiments, the pharmaceutical compositions of the invention are physically stable for at least 1, 2, 3, 4, 5, or 6 months at room temperature, wherein the average size of the particles does not increase by more than about 100% by at least 1, 2, 3, 4, 5, or 6 months. In certain embodiments, the average size of the particles does not increase by more than about 10%, 20%, 30%, 40%, 50%, 60%, 70%, 80%, or 90%.

In certain embodiments, the average particle size of the colloidal dispersion of the present invention is less than about 500 nm, 400 nm, 300 nm, 250 nm, 200 nm, 150 nm, or 100 nm.

This invention also relates to a method of using the pharmaceutical composition for treating various forms of cancer (e.g., through injections), wherein the active anticancer component is alpha-tocopheryl succinate, its analogues or salts, or the combination of alpha-tocopheryl succinate, its analogues or salts and one or more other anticancer agents, including a taxoid analog (e.g., paclitaxel and docetaxel).

### DETAILED DESCRIPTION OF THE INVENTION

The present invention provides stable alpha-tocopheryl succinate colloidal dispersions or dry solid that may be re-hydrated into colloidal dispersions. The alpha-tocopheryl succinate colloidal dispersions may comprise alpha-tocopheryl succinate, analogues or salts thereof, and optionally in combination with another anticancer agent; at least one component selected from the group consisting of an oil component, phospholipid, and an antioxidant; and water. The alpha-tocopheryl succinate dry solid may comprise alpha-tocopheryl succinate, analogues or salts thereof, or optionally in combination with another anticancer agent; and at least one component selected from the group consisting of an oil component, phospholipid(s), and an antioxidant; and a cryoprotectant.

In one aspect, the invention is directed to the use of alpha-tocopheryl succinate (or its analogues or salts) as an anticancer therapeutic agent in an oil-in-water emulsion, wherein alpha-tocopheryl succinate constitutes chiefly the oil phase of the emulsion. Alpha-tocopheryl succinate is not a typical lipid oil: It has a higher polarity than most lipid oils, particularly triglycerides, and is not saponifiable. The hydrophilicity of alpha-tocopheryl succinate is highly pH dependent. At a low pH, generally below pH 5, the end carboxylic acid group of the succinic acid of alpha-tocopheryl succinate is protonated, and alpha-tocopheryl succinate remains a highly hydrophobic solid and does not disperse in water well. At a pH above 5, alpha-tocopheryl succinate is more hydrophilic as the end carboxylic acid group of the succinic acid becomes deprotonated. The deprotonated alpha-tocopheryl succinate is not soluble in water, but behaves like a surfactant of low (Hydrophile-Liphophile Balance) HLB value in water. Thus, upon agitation, the deprotonated alpha-tocopheryl succinate forms an oil-in-water emulsion wherein the oil phase is chiefly alpha-tocopheryl succinate. However, an emulsion formed by alpha-tocopheryl succinate alone (as described in Cancer Lett. 192: 19-24, 2003) appeared to have insufficient stability due to the chemical degradation of alpha-tocopheryl succinate and aggregation of the oil droplets. The present invention provides a new emulsion of alpha-tocopheryl succinate with enhanced stability by the addition of at least one component selected from the group consisting of an oil component, phospholipid, and antioxidant, wherein the alpha-tocopheryl succinate may be in either the deprotonated or protonated form, or a mixture thereof.

In another aspect, the invention is directed to the use of alpha-tocopheryl succinate (or its analogues or salts) as the anticancer therapeutic agent in a solid-in-water suspension, wherein alpha-tocopheryl succinate constitutes chiefly the solid phase of the suspension. In its protonated form, alpha-tocopheryl succinate remains as a solid form and does not form a stable suspension. This invention provides a new alpha-tocopheryl succinate suspension with enhanced stability by the addition of at least one component selected from the group consisting of an oil component, phospholipid, and antioxidant.

In another aspect, the invention comprises an oil-in-solid colloidal dispersion containing alpha-tocopheryl succinate (or its analogues or salts) in the oil droplets, wherein the solid continuous phase is chiefly a cryoprotectant and substantially free of water. In certain embodiments, the water content of an oil-in-solid colloidal dispersion is about, less than about, 10%, 9%, 8%, 7%, 6%, 5%, 4%, 3%, 2% or 1% of the total weight. Upon addition of water or an aqueous medium, the oil-in-solid colloidal dispersion forms an oil-in-water emulsion with the average oil droplet size less than about 5 micron, in certain embodiments, about or less than about 500 nm, 400 nm, 300 nm, 250 nm, 200 nm, 150 nm, or 100 nm.

In another aspect, the invention provides a solid-in-solid colloidal dispersion containing alpha-tocopheryl succinate (or its analogues or salts) in the solid particles, wherein the solid continuous phase is chiefly a cryoprotectant and substantially free of water. In certain embodiments, the water content of the solid-in-solid colloidal dispersion is less than about 10%, 9%, 8%, 7%, 6%, 5%, 4%, 3%, 2% or 1% of the total weight. Upon addition of water or an aqueous medium, the said solid-in-solid colloidal dispersion forms a solid-in-water suspension with the average particle size less than 5 micron, in certain embodiments, less than about 500 nm, 400 nm, 300 nm, 250 nm, 200 nm, 150 nm, or 100 nm. In certain embodiments, the concentration of alpha-tocopheryl succinate (or its analogue or salt) in the solid-in-solid colloidal dispersion may be about 1% to about 30%, about 2% to about 20%, or about 5% to about 15%, by weight. In certain embodiments, the concentration of the dispersed solid component in the solid-in-solid colloidal dispersion may be about 1% to about 20%, about 2% to 15%, or about 3% to 5%, by weight.

“Concentration by weight,” as used herein, refers to the ratio (in percentage) of the weight of a component (e.g., alpha-tocopheryl succinate) of a composition (e.g., a colloidal suspension) to the total weight of the composition, if not otherwise noted.

The colloidal dispersions of the invention for intravenous injection have an average particle size of about 10 to about 1000 nm. In certain embodiments, the average particle size is about 10 to about 500 nm, about 10 nm to about 200 nm, or about 50 to about 150 nm. In certain embodiments, the average particle size is about, or less than about 50 nm, 75 nm, 100 nm, 125 nm, 150 nm, 175 nm, 200 nm, 300 nm, 400 nm, 500 nm, 600 nm, 700 nm, 800 nm, 900 nm, or 1000 nm.

Alpha tocopherol is (±)-(2RS,4′RS,8′RS)-2,5,7,8-tetramethyl-2-(4′,8′, 12′-trimethyltridecyl)-6-chromanol. Its nonproprietary names include Alpha Tocopherol by British Pharmacopoeia, a-Tocopherolum by PhEur, and Vitamin E by the United State Pharmacopoeia. Its CAS Registry Number is 10191-41-0. Its empirical formula is C₂₉H₅₀0₂ and molecular weight is 430.69. The structures of alpha tocopherol and its homologues are shown below.

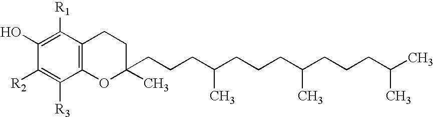

R₁, R₂, and R₃ may be H or CH₃.

| Homologues | R₁ | R₂ | R₃ |
|---|---|---|---|
| Alpha-tocopherol | CH₃ | CH₃ | CH₃ |
| Beta-tocopherol | CH₃ | H | CH₃ |
| Gamma-tocopherol | H | CH₃ | CH₃ |
| Delta-tocopherol | H | H | CH₃ |

The naturally occurring form is known as d-alpha tocopherol or simply alpha tocopherol. Alpha tocopherol has three chrial centers giving rise to eight isomers. The d-isomeric form represents the (2R,4′R,8′R)-alpha-tocopherol or sometimes, RRR-alpha-tocopherol.

“Alpha-tocopheryl succinate” refers to a hemi-ester of succinic acid with alpha tocopherol, such as d-alpha-tocopheryl acid succinate (C₃₃H₅₄O₅, MW 530.8, CAS number 4345-03-3). The chemical structures of alpha-tocopheryl succinate and its analogues are shown below. 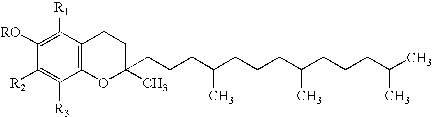 R₁, R₂, R₃═H or CH₃
R═—OOC—(CH₂)ₙ—COOH

| Analogues | n | Dicarboxylic acids |
|---|---|---|
| Alpha-tocopheryl oxalate | 0 | Oxalic acid |
| Alpha-tocopheryl malonate | 1 | Malonic acid |
| Alpha-tocopheryl succinate | 2 | Succinic acid |
| Alpha-tocopheryl glutarate | 3 | Glutaric acid |
| Alpha-tocopheryl adipate | 4 | Adipic acid |
| Alpha-tocopheryl pimelate | 5 | Pimelic acid |
| Alpha-tocopheryl suberate | 6 | Suberic acid |
| Alpha-tocopheryl azelate | 7 | Azelaic acid |

“Alpha-tocopheryl succinate,” in certain embodiments, may include isomers such as dl-alpha-tocopheryl acid succinate (CAS number 17407-37-3). It may, in certain embodiments, include beta tocopheryl acid succinate, delta tocopheryl acid succinate, gamma tocopheryl acid succinate, or isomers thereof.

The term of “alpha-tocopheryl succinate analogues” used in this invention refers to hemi-esters of short-chain dicarboxylic acids with alpha tocopherol, wherein the dicarboxylic acids have the general type formula:
HOOC—(CH₂)ₙ—COOH

Short-chain dicarboxylic acids include oxalic acid (n=0), malonic acid (n=1), succinic acid (n=2), glutaric acid (n=3), adipic acid (n=4), pimelic acid (n=5), suberic acid (n=6), and azelaic acid (n=7) acids.

Alpha-tocopheryl succinate analogues useful in the present invention generally have anticancer activity (i.e., the ability to inhibit cancer growth or cause cancer cell death). In certain embodiments, the anticancer activity of an alpha-tocopheryl succinate analogue is statistically higher than that of alpha-tocopheryl succinate.

The term of “alpha-tocopheryl succinate salts” of this invention refers to an ionic ion salt of pharmaceutically acceptable inorganic counter ions (e.g., sodium, potassium, lithium, calcium, magnesium, and aluminum) and organic counter ions (e.g., amines, lysine, and arginine). Alpha-tocopheryl succinate salts useful in the present invention generally have anticancer activity.

Alpha-tocopheryl succinate, a hemi-ester of alpha tocopherol, structurally and functionally differs from the other three common types of vitamin E derivatives: tocopherol, tocopherol monoester (e.g., acetate), and tocopherol polyetheleneglycol succinate (also referred to as tocopherol PEG ester or vitamin E TPGS). The hemi-esters contain an open (non-esterified) carboxylic acid group and are ionizable, whereas all the others are non-ionizable. Thus, when included as a component in a formulation, the hemi-esters function very different from the monoesters or the parent tocopherol. While a monoester or the parent tocopherol is lipophilic and oil soluble, the hemi-esters are not soluble in either water or oil and are not good solvent or solubilizer for either hydrophilic or hydrophobic drugs. When the open (non-esterified) carboxylic acid group on a hemiester is ionized at a pH about 7 or above, the hemi-esters behave like a surfactant of low HLB value (i.e. water insoluble type) and yet they are not good surfactants like vitamin E TPGS. For example, unlike vitamin E TPGS, tocopherol succinate is incapable of solubilizing a lipophilic drug by forming micelles in water, or emulsifying an vegetable oil in water to form a stable oil-in-water emulsion. By appearance, tocopherol succinate is a crystalline solid, whereas tocopherol and tocopherol acetate are oily liquid, and vitamin E TPGS is a water-soluble wax-like material.

In certain embodiments, the formulations of the present invention does not comprise both alpha-tocopherol and vitamin E TPGS, or either of them.

In certain embodiments, the concentration of alpha-tocopheryl succinate (or its analogue or salt) in a colloidal suspension of the present invention is about 1% to 20% by weight. In certain embodiments, the concentration is about 2% to 15%, or 5% to 10% by weight. In certain embodiments, the concentration is about 1%, 2%, 3%, 4%, 5%, 6%, 7%, 8%, 9%, 10%, 11%,12%,13%, 14%, 15%, 16%, 17%, 18%, 19%, or 20% by weight.

The term “oil” is used herein in a general sense to identify hydrocarbon derivatives, carbohydrate derivatives, or similar organic compounds that are liquid at body temperatures, e.g., about 37° C., and are pharmacologically acceptable in injectable formulations. It includes glycerides or non-glycerides.

The term “oil component” refers to an oil, or a combination of multiple oils in a colloidal dispersion or a dry solid, which may be re-hydrated into a colloidal dispersion. This term does not include alpha-tocopheryl succinate, its analogues, or the salts of alpha-tocopheryl succinate or its analogues.

In certain embodiments, the oil component of a colloidal dispersion or dry solid of the present invention comprises a monoglyceride, a diglyceride, a triglyceride, or a mixture thereof. In certain embodiments, the oil component comprises an ester formed between one or more fatty acids and an alcohol other than glycerol.

“Vegetable oil” refers to oil derived from plant seeds or nuts. Exemplary vegetable oils include, but are not limited to, almond oil, borage oil, black currant seed oil, corn oil, safflower oil, soybean oil, sesame oil, cottonseed oil, peanut oil, olive oil, rapeseed oil, coconut oil, palm oil, canola oil, etc.

Vegetable oils are typically “long-chain triglycerides,” formed when three fatty acids (usually about 14 to about 22 carbons in length, with unsaturated bonds in varying numbers and locations, depending on the source of the oil) form ester bonds with the three hydroxyl groups on glycerol. In certain embodiments, vegetable oils of highly purified grade (also called “super refined”) are generally used to ensure safety and stability of oil-in-water emulsions. In certain embodiments, hydrogenated vegetable oils, which are produced by controlled hydrogenation of the vegetable oil, may be used in the present invention.

“Medium chain triglycerides” (MCT's) is another class of triglyceride oil that can be either naturally derived or synthetic. MCT's are made from fatty acids that are usually about 8 to about 12 carbons in length. Like vegetable oils, MCT's have been used extensively in emulsions designed for injection as a source of calories, for patients requiring parenteral nutrition. Such oil is commercially available as Miglyol 812 from SASOL GmbH, Germany, CRODAMOL GTCC-PN from Croda Inc. of Parsippany, N.J., or Neobees M-5 oil from PVO International, Inc., of Boonton, N.J. Other low-melting medium chain oils may also be used in the present invention.

“Animal fat” refers to oil derived from an animal source. It also comprises triglycerides, but the lengths of, and unsaturated bonds in, the three fatty acid chains vary, compared to vegetable oils. Animal fats from sources that are solid at room temperature (such as tallow, lard, etc.) can be processed to render them liquid if desired. Other types of animal fats that are inherently liquid at room temperature include various fish oils, etc.

In certain embodiments, the combinations of vegetable oil and MCT oil are used in the present invention. Such combinations generally have long record of safe use in combination in injectable emulsions and provide the superior stability for the colloidal dispersions or dry solid of this invention. The specific type of vegetable oil used (i.e., soy bean oil, corn oil, or safflower oil, etc.) is not critical, so long as it is safe, well tolerated, pharmaceutically acceptable, and chemically stable and provides dispersion droplets having a desired size range.

The content of the total oil component in the colloidal suspensions of this invention may be within a range of about 1% to about 20%, by weight. In certain embodiments, the total concentration of the oil component is within a range of about 2% to about 10%, or about 3% to about 5%. In certain embodiments, the total concentration of the oil component is about, or at most about 1%, 2%, 3%, 4%, 5%, 6%, 7%, 8%, 9%, 10%, 12%, 15%, 17%, or 20% by weight. In certain embodiments, the colloidal suspensions comprise oil in an amount that does not result in hyperlipodemia when administered to a subject.

In certain embodiments, the vegetable oil to MCT oil ratio in a colloidal suspension is within a range of about 5:1 to about 1:5, by weight. In certain embodiments, the ratio of the vegetable oil to MCT oil is within a range of about 2:1 to about 1:2. In certain embodiments, the ratio of the vegetable oil to MCT oil is about 5:1, 4:1, 3:1, 2:1, 1:1, 1:2, 1:3, 1:4 or 1:5.

The non-glycerides referred in this invention are chiefly cholesterol and derivatives thereof.

In certain embodiments, the oil component of a formulation of the present invention comprises soybean oil and cholesterol.

In certain embodiments, the ratio of alpha-tocopheryl succinate (or its analogue or salt) to the oil component (e.g., triglyceride or cholesterol) in the colloidal dispersion of this invention is from about 5:1, 4:1, 3:1, 2:1, 1:1, 1:2, 1:3, 1:4, or 1:5.

A “phospholipid” refers to a triester of glycerol with two fatty acids and one phosphate ion. Exemplary phospholipids useful in the present invention include, but are not limited to, phosphatidyl chlorine, lecithin (a mixture of choline ester of phosphorylated diacylglyceride), phosphatidylethanolamine, phosphatidylglycerol, phosphatidic acid with about 4 to about 22 carbon atoms, and more generally from about 10 to about 18 carbon atoms and varying degrees of saturation. The phospholipid component of the drug delivery composition can be either a single phospholipid or a mixture of several phospholipids. The phospholipids should be acceptable for the chosen route of administration.

The phospholipids useful in the present invention can be of natural origin. Naturally occurring phospholipids include soy lecithin, egg lecithin, hydrogenated soy lecithin, hydrogenated egg lecithin, sphingosine, gangliosides, and phytosphingosine and combinations thereof.

Naturally occurring lecithin is a mixture of the diglycerides of stearic, palmitic, and oleic acids, linked to the choline ester of phosphoric acid, commonly called phosphatidylcholine, and can be obtained from a variety of sources such as eggs and soya beans. Soy lecithin and egg lecithin (including hydrogenated versions of these compounds) have a long history of safety, possess combined emulsification and solubilization properties, and tend to be broken down into innocuous substances more rapidly than most synthetic surfactants. Commercially available soya phospholipids are the Centrophase and Centrolex products marketed and sold by Central Soya, Phospholipon from Phospholipid GmbH, Germany, Lipoid by Lipoid GmbH, Germany, and EPIKURON by Degussa.

Hydrogenated lecithin is the product of controlled hydrogenation of lecithin. It may also be used in the present invention.

According to the United State Pharmacopoeia (USP), lecithin is a non-proprietary name describing a complex mixture of acetone-insoluble phospholipids, which consists chiefly of phosphotidylcholine, phosphotidylethanolamine, phosphotidylserine and phosphotidylinositol, combined with various amounts of other substances such as triglycerides, fatty acids, and carbohydrates.

Pharmaceutically, lecithins are mainly used as dispersing, emulsifying, and stabilizing agents and are included in intramuscular and intravenous injections, parenteral nutritional formulations and topical products. Lecithin is also listed in the FDA Inactive Ingredients Guide for use in inhalations, IM and IV injections, oral capsules, suspensions and tablets, rectal, topical, and vaginal preparations.

Phospholipids can also be synthesized and the common synthetic phospholipids are listed below:

Diacylglycerols

1,2-Dilauroyl-sn-glycerol (DLG)
1,2-Dimyristoyl-sn-glycerol (DMG)
1,2-Dipalmitoyl-sn-glycerol (DPG)
1,2-Distearoyl-sn-glycerol (DSG)
   Phosphatidic Acids
1,2-Dimyristoyl-sn-glycero-3-phosphatidic acid, sodium salt (DMPA,Na)
1,2-Dipalmitoyl-sn-glycero-3-phosphatidic acid, sodium salt (DPPA,Na)
1,2-Distearoyl-sn-glycero-3-phosphatidic acid, sodium salt (DSPA,Na)
   Phosphocholines
1,2-Dilauroyl-sn-glycero-3-phosphocholine (DLPC)
1,2-Dimyristoyl-sn-glycero-3-phosphocholine (DMPC)
1,2-Dipalmitoyl-sn-glycero-3-phosphocholine (DPPC)
1,2-Distearoyl-sn-glycero-3-phosphocholine (DS PC)
   Phosphoethanolamines
1,2-Dilauroyl-sn-glycero-3-phosphoethanolamine (DLPE)
1,2-Dimyristoyl-sn-glycero-3-phosphoethanolamine (DMPE)
1,2-Dipalmitoyl-sn-glycero-3-phosphoethanolamine (DPPE)
1,2-Distearoyl-sn-glycero-3-phosphoethanolamine (DSPE)
   Phosphoglycerols
1,2-Dilauroyl-sn-glycero-3-phosphoglycerol, sodium salt (DLPG)
1,2-Dimyristoyl-sn-glycero-3-phosphoglycerol, sodium salt (DMPG)
1,2-Dimyristoyl-sn-glycero-3-phospho-sn-1-glycerol, ammonium salt (DMP-sn-1-G,NH₄)
1,2-Dipalmitoyl-sn-glycero-3-phosphoglycerol, sodium salt (DPPG,Na)
1,2-Distearoyl-sn-glycero-3-phosphoglycerol, sodium salt (DSPG,Na)
1,2-Distearoyl-sn-glycero-3-phospho-sn-1-glycerol, sodium salt (DSP-sn-1 G,Na)
   Phosphoserines
1,2-Dipalmitoyl-sn-glycero-3-phospho-L-serine, sodium salt (DPPS,Na)
Mixed Chain Phospholipids
1-Palmitoyl-2-oleoyl-sn-glycero-3-phosphocholine (POPC)
1-Palmitoyl-2-oleoyl-sn-glycero-3-phosphoglycerol, sodium salt (POPG,Na)
1-Palmitoyl-2-oleoyl-sn-glycero-3-phosphoglycerol, ammonium salt (POPG,NH₄)
   Lysophospholipids
1-Palmitoyl-2-lyso-sn-glycero-3-phosphocholine (P-lyso-PC)
1-Stearoyl-2-lyso-sn-glycero-3-phosphocholine (S-lyso-PC)
   Pegylated Phospholipids
N-(Carbonyl-methoxypolyethyleneglycol 2000)-MPEG-2000-DPPE
1,2-dipalmitoyl-sn-glycero-3-phosphoethanolamine, sodium salt
N-(Carbonyl-methoxypolyethyleneglycol 5000)-MPEG-5000-DSPE
1,2-distearoyl-sn-glycero-3-phosphoethanolamine, sodium salt
N-(Carbonyl-methoxypolyethyleneglycol 5000)-MPEG-5000-DPPE
1,2-dipalmitoyl-sn-glycero-3-phosphoethanolamine, sodium salt
N-(Carbonyl-methoxypolyethyleneglycol 750)-MPEG-750-DS PE
1,2-distearoyl-sn-glycero-3-phosphoethanolamine, sodium salt
N-(Carbonyl-methoxypolyethyleneglycol 2000)-MPEG-2000-DSPE
1,2-distearoyl-sn-glycero-3-phosphoethanolamine, sodium salt

The amount of phospholipids, by weight, in the colloidal suspensions or dry solid of this invention may be within a range of about 0.5% to about 10%. In certain embodiments, the amount of phospholipids, by weight, may be within a range of about 1% to about 5%, or about 2% to about 3%. In certain embodiments, the amount of phospholipids is about 0.5%, 1%, 2%, 3%, 4%, 5%, 6%, 7%, 8%, 9% or 10%.

In certain embodiments, the ratio of alpha-tocopheryl succinate to phospholipid in the colloidal dispersion of this invention is from about 5:1 to about 1:5 (w/w). In certain embodiments, the ratio is about 5:1, 4:1, 3:1, 2:1, 1:1,1:2, 1:3, 1:4, or 1:5.

A “colloidal dispersion” refers to a system in which particles of colloidal size of any nature (e.g., solid, liquid or gas) are dispersed in a continuous phase of a different composition (or state).

The term “colloidal” refers to a state of subdivision, implying that the molecules or polymolecular particles dispersed in a medium have at least, in one direction, a dimension roughly between 1 nm and 1 mm, or that in a given system, discontinuities are found at distances of that order (1972, 31, 605, IUPAC Compendium of Chemical Terminology, 2nd Edition,1997).

In certain embodiments, a colloidal dispersion of this invention is an emulsion, i.e., a colloidal dispersion of a water-immiscible liquid dispersed in an aqueous medium (“oil-in-water type colloidal dispersion”). The water-immiscible liquid is in a form of oil droplets comprising alpha-tocopheryl succinate and optionally another therapeutic agent and/or other water insoluble components, whose diameter are generally between about 0.1 and about 3.0 microns. The emulsion is typically optically opaque unless the dispersed and continuous phases are refractive index matched. Such systems possess a finite stability, generally defined by the application or relevant reference system, which may be enhanced by the addition at least one component selected from a group consisting of an oil component, phospholipid, and antioxidant.

In certain other embodiments, a colloidal dispersion of this invention is a suspension in an aqueous medium (“solid-in-water type colloidal dispersion”). The suspension of this invention is a colloidal dispersion of a water-insoluble solid phase comprising alpha-tocopheryl succinate and optionally another therapeutic agent and/or other water insoluble components in the form of small particles whose diameters are generally between 0.1 and 3.0 microns. The suspension is typically optically opaque unless the dispersed and continuous phases are refractive index matched. Such systems possess a finite stability, generally defined by the application or relevant reference system, which may be enhanced by the addition at least one component selected from a group consisting of an oil component, phospholipid, and antioxidant.

In certain other embodiments, a colloidal dispersion of this invention is a dispersion of oil droplets in a solid continuous phase (oil-in-solid type colloidal dispersion). The oil droplets comprise alpha-tocopheryl succinate and optionally another therapeutic agent and/or other water insoluble components. The continuous phase is a solid matrix comprising primarily a cryo-protectant. This colloidal dispersions may be formed by freeze-drying or spray drying an oil-in-water type colloidal dispersion. Such oil-in-solid systems possess an enhanced stability compared to the oil-in-water colloidal dispersion, generally due to the removal of water from the continuous phase.

In certain other embodiments, a colloidal dispersion of this invention is a dispersion of solid particles in a solid continuous phase (“solid-in-solid type colloidal dispersion”). The solid particles comprise alpha-tocopheryl succinate and optionally another therapeutic agent and/or other water insoluble components. The continuous phase is a solid matrix comprising primarily a cryo-protectant. This colloidal dispersion may be formed by freeze-drying or spray drying a solid-in-solid type colloidal dispersion. Such solid-in-solid systems possess an enhanced stability compared to the solid-in-water colloidal dispersion, generally due to the removal of water from the continuous phase. “Aqueous medium” or “aqueous phase” refers to a water-containing liquid which can contain pharmaceutically acceptable additives, such as acidifying, alkalizing, buffering, chelating, complexing and solubilizing agents, antioxidants and antimicrobial preservatives, suspending and/or viscosity modifying agents, tonicity modifying agent, cryo-protectant, and other biocompatible materials or therapeutic agents. In certain embodiments, such additives assist in stabilizing the colloidal dispersion or in rendering the formulations of the present invention biocompatible.

The aqueous phase generally has an osmolality of approximately 300 mOsm and may include potassium or sodium chloride, trahalose, sucrose, sorbitol, glycerol, mannitol, polyethylene glycol, propylene glycol, albumin, amino acid and mixtures thereof. In certain embodiments, a tonicity of at least 250 mOsm is achieved with an agent that also increases viscosity, such as sorbitol or sucrose. “Antioxidants” used in this invention refer to primarily metal ion chelator and/or reducing agents that are safe to use in an injectable product. A metal ion chelator works as an antioxidant by binding to metal ions and thereby reduces the catalytic effect of metal ion on the oxidation reaction of alpha-tocopheryl succinate. Metal chelators that are useful in this invention may include EDTA, glycine and citric acid or salts thereof.

In certain embodiments, the concentration of disodium edetate in the colloidal dispersion of this invention can be from about 0.0001% to about 1% w/v. In certain embodiments, the concentration is from about 0.001% to about 0.1% w/v, or from about 0.001% to about 0.005% w/v.

The reducing agents exhibit their antioxidant effect by reacting with oxidizing agents in competition with alpha-tocopheryl succinate or by converting oxidized alpha-tocopheryl succinate back to the original alpha-tocopheryl succinate in the reduced form. The reducing agents useful in this invention include, but are not limited to, ascorbic acid or salts thereof, ascorbyl palmitate, sodium metabisulfite, propyl gallate, butylated hydroxyanisole, butylated hydroxytoluene, tocopherol, amino acids or salts thereof, citric acid or salts thereof, reducing sugars, or mixtures thereof.

A “cryoprotectant” of this invention refers to a safe and biocompatible agent that protects the oil-in-water or solid-in-water colloidal dispersion during freezing or freeze-drying by maintaining the discrete and sub-micron size droplets or particles in the aqueous surrounding. The cryoprotectants of this invention also function as the main component of the continuous phase of the oil-in-solid or solid-in-solid colloidal dispersion. The cryoprotectants useful for this invention include, but are not limited to, monosaccharide, disaccharide, polysaccharide, propylene glycol, polyethylene glycol, glycerol, poly-ol, dextrin, cyclodextrin, starch, cellulose and cellulose derivative, protein, peptide, amino acid, sodium chloride, polyvinypyrrolidone, or mixtures thereof. For instance, in certain embodiments, the cryoprotectant is mannitol, sorbitol, xylitol, lactose, fructose, xylose, sucrose, trahalose, mannose, maltose, dextrose, dexstrane, or a mixture thereof. In certain embodiments, the cryoprotectant is sucrose, a combination of sucrose and mannitol, or a combination of sucrose and trehalose. In certain embodiments, the formulations of the present invention do not comprise acacia.

In certain embodiments, the concentration of cryoprotectants in the oil-in-solid or solid-in-solid formulations of the present invention may be about 30% to 70% by weight. In certain embodiments, the concentration of sucrose may be about 40% to 60% by weight.

The concentration of cryoprotectants in the oil-in-water or solid-in-water colloidal dispersion of this invention may be from about 1% to about 30% w/v. In certain embodiments, the concentration is from about 3% to about 15% w/v or from about 5% to 10% w/v.

“Biocompatible” refers to the capability of performing functions within or upon a living organism in an acceptable manner, i.e., without undue toxicity or physiological or pharmacological effects.

In certain embodiments, the present compositions are both chemically and physically stable. A composition is “chemically stable” if less than about 20% alpha-tocopheryl succinate in the composition is chemically degraded after storage under appropriate conditions for a defined period of time (e.g., a month). In certain embodiments, the concentration of the intact alpha-tocopheryl succinate (or its analogue or salt) in the composition is reduced by less than about 5%, 10%, 15% or 20% under appropriate storage conditions (e.g., at −20° C., 2-8° C., or room temperature) for at least 1, 2, 3, 4, 5, 6, 9, 12, 15, 18, or 24 months.

Chemical degradation of alpha-tocopheryl succinate (or its analogues or salts) includes mainly hydrolysis of the succinate ester bond and oxidation of the tocopherol moiety. Hydrolysis of the succinate ester bond leads to the formation of succinic acid and tocopherol, each of which is inefficacious against cancer and is prone to a fast oxidation. The rate of hydrolysis of alpha-tocopheryl succinate is pH-dependent. The removal of water in the continuous phase by freeze-drying, spray-drying, or other drying means essentially stops the hydrolytic degradation.

Another degradation route of alpha-tocopheryl succinate (or its analogues or salts) is oxidation. Tocopherols are slowly oxidized by atomospheric oxygen and rapidly by metal ions such as ferric and silver salts. Oxidation products include tocopheroxide, tocopherylquinone and tocopherylhydroquinone, or dimers and trimers thereof. Tocopherol esters such as alpha-tocopheryl succinate are more stable to oxidation than free tocopherols. Oxidation may be prevented or reduced by the use of an antioxidant.

A composition (e.g., a colloidal suspension or a dry solid) is “physically stable” if it may be stored under appropriate conditions for a defined period of time (e.g., 1 month) without increase in its average particle size by more than 100%, or evidence of phase separation, creaming, or particle aggregation. In certain embodiments, the average size of particles of a composition of the present invention does not increase by more than about 10%, 20%, 25%, 30%, 40%, 50%, 75%, or 100% under appropriate storage conditions (e.g., at −20° C., 2-8° C., or room temperature) for at least 1, 2, 3, 4, 5, 6, 9, 12, 15, 18, or 24 months.

In certain embodiments, a colloidal dispersion composition of alpha-tocopheryl succinate is capable of retaining no less than 90% of the intact alpha-tocopheryl succinate (or its analogue or salt) and is substantially free from aggregates of greater than 5-micron in diameter for at least 6 months at room temperature. In certain embodiments, a colloidal dispersion composition of alpha-tocopheryl succinate is capable of retaining no less than 92%, 94%, 95%, 96%, 97%, 98% or 99% of the intact alpha-tocopheryl succinate (or its analogue or salt) and is substantially free from aggregates of greater than 2-micron in diameter for at least 6 months at room temperature.

Unless noted otherwise, a pharmaceutical composition is “stable” if the pharmaceutical composition is both chemically and physically stable for a defined period of time.

“Room temperature” refers to temperature ranging from about 20° C. to about 25° C.

“Therapeutic agent” refers to any compound natural or synthetic that has therapeutic effects on a mammal (including human). Therapeutic agents include anticancer agents and may be used in addition to the alpha-tocopheryl succinate or its analogue or salt in the same formulation.

“Chemotherapeutic agents” or “anticancer agents” refer to any natural or synthetic molecules that are effective against one or more forms of cancer (e.g., breast, ovarian, and lung cancer). In certain embodiments, the chemotherapeutic agents are slightly or completely lipophilic (i.e., water insoluble), or can be modified to be lipophilic. Chemotherapeutic agents include molecules that are cytotoxic (anti-cancer agents), that stimulate the immune system (immune stimulators), and that modulate or inhibit angiogenesis.

Chemotherapeutics include, but are not limited to, alkylating agents, antimetabolites, taxanes, cytotoxics, cytoprotectant adjuvants, LHRH analogues, platinum agents, anti-estrognes, anti-androgens, hormonals, aromatase inhibitors, cell cycle controlling agents, apoptosis agents, topoisomerase inhibitors, angiogenesis inhibitors, immunotherapy agents, monoclonal antibodies, retinoid, kinase inhibitors and signal transduction inhibitors.

In certain embodiments, the chemotherapeutic is selected from paclitaxel, docetaxel and related molecules collectively termed taxoids, taxines or taxanes.

In certain embodiments, the chemotherapeutic is selected from podophyllotoxins and their derivatives and analogues.

In certain embodiments, chemotherapeutics useful in this invention are camptothecins.

In certain other embodiments, chemotherapeutics useful in this invention are the lipophilic anthracyclines.

In certain other embodiments, chemotherapeutics useful in this invention are compounds that are lipophilic or can be made lipophilic by molecular chemosynthetic modifications well known to those skilled in the art, for example, by combinatorial chemistry and by molecular modeling. Such chemotherapeutics include: Amonafide, Illudin S, 6-hydroxymethylacylfulvene Bryostatin 1, 26-succinylbryostatin 1, Palmitoyl Rhizoxin, DUP 941, Mitomycin B, Mitomycin C, Penclomedine, interferon alpha.2b, angiogenesis inhibitor compounds (e.g., cisplatin hydrophobic complexes such as 2-hydrazino-4,5-dihydro-1 H-imidazole with platinum chloride and 5-hydrazino-3,4-dihydro-2H-pyrrole with platinum chloride), vitamin A and its derivatives.

Other chemotherpeutics useful in the invention include: 1,3-bis(2-chloroethyl)-1-nitrosurea (“carmustine” or “BCNU”), 5-fluorouracil, doxorubicin (“adriamycin”), epirubicin, aclarubicin, Bisantrene (bis(2-imidazolen-2-ylhydrazone)-9,10-anthracenedicarboxaldehyde, mitoxantrone, methotrexate, edatrexate, muramyl tripeptide, muramyl dipeptide, lipopolysaccharides, 9-b-d-arabinofairanosyladenine (“vidarabine”) and its 2-fluoro derivative, gemcitabine, resveratrol, retinoic acid and retinol, carotenoids, and tamoxifen.

Other chemotherapeutics useful in this invention include: Decarbazine, Lonidamine, Piroxantrone, Anthrapyrazoles, Etoposide, Camptothecin, 9-aminocamptothecin, 9-nitrocamptothecin, camptothecin-11 (“Irinotecan”), Topotecan, Bleomycin, the Vinca alkaloids and their analogs [Vincristine, Vinorelbine, Vindesine, Vintripol, Vinxaltine, Ancitabine], 6-animochrysene, and vinorelbine.

Other chemotherapeutics useful in the present invention are mimetics of taxol, eleutherobins, sarcodictyins, discodermolides and epothiolones.

In certain embodiments, the presence of an anti-cancer agent other than alpha-tocopheryl succinate or its analogue or salt in the pharmaceutical composition of the present invention results in additive or synergistic anticancer activities. In certain other embodiments, the concentration of alpha-tocopheryl succinate or its analogue or salt in the pharmaceutical compositions of the present invention is relatively low (e.g., less than about 3%, 2%, or 1% by weight) so that alpha-tocopheryl succinate or its analogue or salt does not contribute significantly to the anticancer activities of the pharmaceutical compositions. In such embodiments, alpha-tocopheryl succinate or its analogue or salt, however, stabilizes compositions (e.g., oil-in-water emulsions, solid-in-water suspensions, oil-in-solid dispersions, and solid-in-solid dispersions) that comprise another anticancer agent (e.g., docetaxel or paclitaxel) and thus allow for higher loading of the other anticancer agent in, and higher anticancer activities of, such compositions. In certain embodiments, the present invention provides a colloidal dispersion comprises about 1% to about 20% (e.g., about 5% to about 15%) by weight alpha-tocopheryl succinate, an analogue or salt thereof; about 1% to about 20% (e.g., 3% to 5%) by weight an oil component; and optionally 0.005%-0.1% by weigh edetic acid sodium salt in an aqueous medium having a pH at between about 6 and about 8; and optionally an osmotic pressure modifier; wherein the colloidal dispersion has an average particle diameter less than about 200 nm (e.g., less than about 150 nm).

An exemplary colloidal dispersion of the present invention may comprise about about 5% to about 15% by weight alpha-tocopheryl succinate, an analogue or salt thereof; 3% to 5% by weight an oil component; and optionally 0.005%-0.1% by weigh edetic acid sodium salt in an aqueous medium having a pH at between about 6 and about 8; and optionally an osmotic pressure modifier; wherein the colloidal dispersion has an average particle diameter less than about about 150 nm.

In centain embodiments, the present invention a dry solid that comprises about 1% to about 30% (e.g., about 5% to about 15%) by weight alpha-tocopheryl succinate, an analogue or salt thereof; about 1% to about 20% (e.g., about 3% to about 5%) by weight an oil component; about 10% to about 80% by weight cryoprotectant (e.g., sucrose); and optionally about 0.005% to about 1% by weigh edetic acid sodium salt; wherein the dry solid, upon mixing with an aqueous medium, forms a colloidal dispersion having an average particle diameter less than about 200 nm (e.g., less than about 150 nm) and a pH at between about 6 and about 8.

An exemplary dry solid of the present invention may comprise about 5% to about 15% by weight alpha-tocopheryl succinate, an analogue or salt thereof; about 3% to about 5% by weight an oil component; about 10% to about 80% by weight sucrose; and optionally about 0.005% to about 1% by weigh edetic acid sodium salt; wherein the dry solid, upon mixing with an aqueous medium, forms a colloidal dispersion having an average particle diameter less than about 150 nm and a pH at between about 6 and about 8.

In certain embodiments, the present invention provides a dry solid that comprises about 1-15% (e.g., about 11.6%) by weight alpha-tocopheryl succinate, about 15-35% (e.g., 28.3%) by weight lecithin, about 1-5% (e.g., about 3.9%) by weight cholesterol, and about 30-60% (e.g., 56.2%) by weight cryoprotectant (e.g., sucrose); wherein the dry solid, upon mixing with an aqueous medium, forms a colloidal dispersion having an average particle diameter less than about 200 nm and a pH at between about 6 and about 8.

An example of the dry solid of the present invention may comprise about 11.6% by weight alpha-tocopheryl succinate, about 28.3% by weight egg lecithin, about 3.9% by weight cholesterol, and about 56.2% by weight sucrose; wherein the dry solid, upon mixing with an aqueous medium, forms a colloidal dispersion having an average particle diameter less than about 200 nm and a pH at between about 6 and about 8.

In certain embodiments, the present invention provides a solid-in-water colloidal dispersion comprises about 1-5% (e.g., about 3.6%) by weight alpha-tocopheryl succinate, about 6-10% (e.g., 8.8%) by weight lecithin (e.g., egg lecithin), about 0.5-2% (e.g., 1.2%) by weight cholesterol, and about 10-20% (e.g., about 17.5%) by weight cryoprotectant (e.g., sucrose); wherein the colloidal dispersion having an average particle diameter less than about 200 nm and a pH at between about 6 and about 8.

An example of the solid-in-water colloidal dispersions of the present invention may comprise about 3.6% by weight alpha-tocopheryl succinate, about 8.8% by weight egg lecithin, about 1.2% by weight cholesterol, and about 17.5% by weight sucrose; wherein the colloidal dispersion having an average particle diameter less than about 200 nm and a pH at between about 6 and about 8.

In certain embodiments, the present invention provides a dry solid that comprises about 1-15% (e.g., about 11.5%) by weight alpha-tocopheryl succinate, about 15-30% (e.g., about 27.8%) by weight lecithin (e.g., egg lecithin), about 1-5% (e.g., about 3.8%) by weight cholesterol, about 30-60% (e.g., about 55.3%) by weight sucrose, and 0.5-2% (e.g., about 1.6%) by weight paclitaxel or docetaxel; wherein the dry solid, upon mixing with an aqueous medium, forms a colloidal dispersion having an average particle diameter less than about 200 nm and a pH at between about 6 and about 8.

An example of the dry solid of the present invention may comprise about 11.5% by weight alpha-tocopheryl succinate, about 27.8% by weight egg lecithin, about 3.8% by weight cholesterol, about 55.3% by weight sucrose, and about 1.6% by weight paclitaxel or docetaxel; wherein the dry solid, upon mixing with an aqueous medium, forms a colloidal dispersion having an average particle diameter less than about 200 nm and a pH at between about 6 and about 8.

The pharmaceutical compositions of the invention are typically formed by mixing alpha-tocopheryl succinate, an analogue or salt thereof and optionally in combination with another anti-cancer agent; at least one component selected from the group consisting of an oil component, phospholipid, antioxidant, optionally cryoprotectant, and water; adjusting the pH to 5-8; homogenizing to form a uniform colloidal dispersion with an average particle/droplet diameter less than about 1000 nm (e.g., via a mechanical homogenization method including high shear mixing, high pressure extrusion, or microfluidization); passing a sterilizing filter; and optionally drying the colloidal dispersion by a freeze-drying or spray-drying method.

In a related aspect, the present invention provides a method of treating carcinomas comprising administering the colloidal dispersion of this invention to a subject in need of such a treatment. The colloidal dispersions may be administered to animals or humans via intravascular, oral, intramuscular, cutaneous and subcutaneous routes. Other routes of administration include, but are not limited to, intraabdominal, intraarterial, intraarticular, intracapsular, intracervical, intracranial, intraductal, intradural, intralesional, intralocular, intralumbar, intramural, intraocular, intraoperative, intraparietal, intraperitoneal, intrapleural, intrapulmonary, intraspinal, intrathoracic, intratrachcal, intratympanic, intrauterine, and intraventricular. The colloidal dispersions of the present invention may also be nebulized using suitable aerosol propellants, which are known in the art for pulmonary delivery of lipophilic compounds.

The general principles of the present invention may be more fully appreciated by reference to the following non-limiting examples.

### EXAMPLES

### Example 1

A 5% alpha-tocopheryl succinate dispersion was prepared according to the conditions described in Cancel Lett. 192: 19-24, 2003 in the following composition:

| | |
|---|---|
| Alpha-tocopheryl succinate | 5% w/w |
| De-ionized water to QS | |
| NaOH to adjust pH to 7.0 | |

The process included the following steps:

(1) Weigh out alpha-tocopheryl succinate (Vitamin E succinate, Product No. V 1176 by Spectrum Chemicals) 4.25 mg,

(2) Add de-ionized water 56 g,

(3) Adjust pH to 7.1 using 1N NaOH solution while stirring,

(4) Add more de-ionized water to a final concentration of alpha-tocopheryl succinate of 5% w/w, and

(5) Apply vigorous mechanical agitation using an Ultra-Turrax high-shear mixer to obtain a white, opaque and uniform dispersion.

The average particle diameter was measured using a laser light scattering spectrometer (LLS, Model 370 by Particle Sizing Systems, Santa Barbara, Calif.) to be 235 nm. This dispersion was kept in an airtight glass container in a 5° C. refrigerator for 2 weeks. Noticeable aggregation was observed in the dispersion such as curd-like precipitates and gel-like coating adhering to the glass wall.

It is thus concluded that the alpha-tocopheryl succinate composition according to Cancer Lett. 192: 19-24, 2003 is physically unstable and not suited as an injectable product for human use.

### Example 2

A 5% alpha-tocopheryl succinate dispersion was prepared according to the following composition:

| | |
|---|---|
| Alpha-tocopheryl succinate | 5% w/w |
| Soybean oil | 4% w/w |
| Medium chain triglyceride | 4% w/w |
| De-ionized water to QS | |
| NaOH to adjust pH to 7.0 | |

The process included the following steps:

(1) Prepare a alpha-tocopheryl succinate dispersion using the same procedure as in the Example 1,

(2) Add soybean oil and medium chain triglyceride to the alpha-tocopheryl succinate dispersion, and

(3) Mixing by vigorous agitation using a Mini Beadbeater (BioSpec) to obtain a white, opaque and uniform dispersion.

The average droplet diameter was measured using a laser light scattering spectrometer (Model 370 by Particle Sizing Systems, Santa Barbara, Calif.) to be 105 nm. This dispersion was kept in an airtight glass container in a 5° C. refrigerator for 2 weeks. No sign of degradation or aggregation was observed.

It is thus concluded that a alpha-tocopheryl succinate composition prepared according to the present invention that contains an oil component (soybean oil and medium chain triglyceride) is more stable than the alpha-tocopheryl succinate dispersion described in Example 1 according to Cancer Letter 192:19-24, 2003.

### Example 3

A 5% alpha-tocopheryl succinate dispersion was prepared according to the following composition:

| | |
|---|---|
| Alpha-tocopheryl succinate | 5% w/w |
| Soybean oil | 2.5% w/w |
| Soy lecithin (LIPOID S-100) | 2.5% w/w |
| De-ionized water to QS | |
| NaOH to adjust pH to 7.0 | |

The process included the following steps:

(1) Prepare a alpha-tocopheryl succinate dispersion using the same procedure as in the Example 1,

(2) Add soybean oil and Soy lecithin to the alpha-tocopheryl succinate dispersion, and

(3) Mixing by vigorous agitation using a Mini Beadbeater (BioSpec) to obtain a white, opaque and uniform dispersion.

The average droplet diameter was measured using a laser light scattering spectrometer (Model 370 by Particle Sizing Systems, Santa Barbara, Calif.) to be 213 nm. This dispersion was kept in an airtight glass container in a 5° C. refrigerator for 2 weeks. No sign of degradation or aggregation was observed.

It is thus concluded that a alpha-tocopheryl succinate composition according to this invention that contains an oil component (soybean oil) and phospholipid (soy lecithin) is physically and chemically more stable than the alpha-tocopheryl succinate dispersion described Example 1 according to Cancer Lett. 192:19-24, 2003.

### Example 4

The dispersions prepared in Example 1, 2 and 3 were compared for their capability of carrying an insoluble anti-cancer agent, i.e., paclitaxel.

The study was conducted as follow:

(1) Into each dispersion (1000 mg) add 0.5 mg Paclitaxel (SiChuan KangYi Corp, China);

(2) Mixing by vigorous agitation using a Mini Beadbeater (BioSpec) to obtain a white, opaque and uniform dispersion and then rotate the dispersion at room temperature for 16 hours,

(3) Filter each dispersion through a 0.2 micron size syringe filter, and

(4) Dilute the filtrate and perform an HPLC analysis for Paclitaxel concentration in the filtrate.

Results

| Formulation | Paclitaxel concentration (mg/mL) |
|---|---|
| Dispersion in Example 1 | 0.19 |
| Dispersion in Example 2 | 0.39 |
| Dispersion in Example 3 | 0.26 |

It is thus concluded that alpha-tocopheryl succinate compositions according to this invention, i.e., with an oil component (Example 2) or with both oil component and phospholipid (Example 3), are capable of carrying significantly more insoluble anticancer drug Paclitaxel than the alpha-tocopheryl succinate dispersion described in Example 1 according to Cancer Lett. 192: 19-24, 2003, while maintaining good stability.

### Example 5

Another alpha-tocopheryl succinate dispersion according to this invention was prepared to contain 3.6% alpha-tocopheryl succinate, and optionally the insoluble anticancer drug Paclitaxel according to the following compositions:

| | |
|---|---|
| Alpha-tocopheryl succinate | 3.6% w/w |
| Paclitaxel | 0.5% w/w |
| | (optional) |
| Egg lecithin (LIPOID E80) | 8.8% w/w |
| Cholesterol | 1.2% w/w |
| Sucrose | 17.5% w/w |
| De-ionized water to QS | |
| NaOH/HCl to adjust pH to 7.0 | |

The preparation of an oil phase was performed as follows:

(1) Weigh out, egg lecithin and cholesterol, and optionally paclitaxel or docetaxel all in one container,

(2) Add enough anhydrous ethanol to dissolve all solids and obtain a clear yellow solution, and

(3) Apply rotary vacuum drying to remove ethanol completely to obtain a semi-solid oil phase.

The preparation of a alpha-tocopheryl succinate colloidal dispersion was performed as follows:

(1) Weigh out the oil phase, sucrose and alpha-tocopheryl succinate, all in one container,

(2) Apply a vigorous mechanical agitation using an Ultra-Turrax high-shear mixer for 5 minutes to obtain a crude colloidal dispersion,

(3) Pass the crude colloidal dispersion through a microfluidizer (Model 110F by Microfluidics, Mass.) operating at 18000 psi pressure six times to obtain a translucent, slightly yellow colloidal dispersion, and

(4) Filter the microfluidized dispersion through a 0.2 μm membrane filter.

The average colloidal dispersion droplet diameter was measured using a laser light scattering spectrometer (Model 370 by Particle Sizing Systems, Santa Barbara, Calif.) to be 120-130 nm.

The preparation of a alpha-tocopheryl succinate solid-in-solid dispersion by freeze-drying was performed as follows:

(1) The filtered colloidal dispersion was filled at 0.9 mL into each 2 mL glass vial and was freeze-dried using a Virtis Advantage Freeze-drier to form uniform white mass (a solid-in-solid dispersion), and

(2) Add deionized water to the solid-in-solid dispersion and mix gently to obtain a translucent slightly yellow dispersion (a solid-in-water dispersion).

The average droplet diameter of the reconstituted alpha-tocopheryl succinate dispersion was measured using a laser light scattering spectrometer (Model 370 by Particle Sizing Systems, Santa Barbara, Calif.) to be 120-130 nm. The alpha-tocopheryl succinate solid-in-solid dispersion was kept in an airtight glass container in a 5° C. refrigerator for 4 weeks. No degradation and aggregation was observed.

It is thus concluded that a solid-in-solid alpha-tocopheryl succinate colloidal dispersion according to this invention can be prepared with optionally an insoluble anticancer drug Paclitaxel. The compositions prepared in this example are stable.

### Example 6

### Hemolysis Test

Two alpha-tocopheryl succinate dispersions were prepared according to this invention to contain the following compositions:

| | |
|---|---|
| Alpha-tocopheryl succinate | 5% w/w |
| Egg lecithin (LIPOID E80) | 5% w/w |
| De-ionized water to QS | |
| NaOH/HCl to adjust pH to 7.0 | |
| Alpha-tocopheryl succinate | 5% w/w |
| Soybean oil | 2.5% w/w |
| Egg lecithin (LIPOID E-80) | 2.5 w/w |
| De-ionized water to QS | |
| NaOH/HCl to adjust pH to 7.0 | |

Both dispersions were tested for hemolysis using rabbit blood. Rabbit red blood cells (2%) were suspended in normal saline and were mixed with the test articles as follow:

| | Test tube | | | | | | |
|---|---|---|---|---|---|---|---|
| | 1 | 2 | 3 | 4 | 5 | 6 | 7 |
| 2% RBC suspension, mL | 2.5 | 2.5 | 2.5 | 2.5 | 2.5 | 2.5 | 2.5 |
| Normal saline | 2.4 | 2.3 | 2.2 | 2.1 | 2.0 | 2.5 | 0 |
| (negative control), mL | | | | | | | |
| alpha-tocopheryl | 0.1 | 0.2 | 0.3 | 0.4 | 0.5 | 0 | 0 |
| succinate dispersion, | | | | | | | |
| mL | | | | | | | |
| Distilled water | 0 | 0 | 0 | 0 | 0 | 0 | 2.5 |
| (Positive control), mL | | | | | | | |

The test tubes were kept at 37° C. and were observed for hemolysis (when the supernatant becomes red). Test tube 7 showed hemolysis. Neither alpha-tocopheryl succinate dispersion showed sign of hemolysis in 2 hours. It is concluded that the alpha-tocopheryl succinate dispersions according to this invention are not hemolytic and are suitable for injection.

All of the above U.S. patents, U.S. patent application publications, U.S. patent applications, foreign patents, foreign patent applications and non-patent publications referred to in this specification and/or listed in the Application Data Sheet, are incorporated herein by reference, in their entirety.

From the foregoing it will be appreciated that, although specific embodiments of the invention have been described herein for purposes of illustration, various modifications may be made without deviating from the spirit and scope of the invention. Accordingly, the invention is not limited except as by the appended claims.

1. A pharmaceutical composition, comprising alpha-tocopheryl succinate, an analogue or salt thereof; at least one component selected from the group consisting of an oil component, phospholipid, and antioxidant; and water; wherein the composition is a colloidal dispersion having an average particle size less than 200 nm in diameter, the alpha-tocopheryl succinate, the analogue or salt thereof is stable for at least 6 months at room temperature, and the alpha-tocopheryl succinate has the chemical structure: 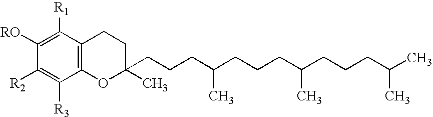
wherein R₁ is CH₃, R₂ is CH₃, R₃ is CH₃, and R is —OOC—(CH₂)₂—COOH.

2. A pharmaceutical composition, comprising alpha-tocopheryl succinate, an analogue or salt thereof; at least one component selected from the group consisting of an oil component, phospholipid, and antioxidant; and a cryoprotectant; wherein the composition is a dry solid, the alpha-tocopheryl succinate, the analogue or salt thereof is stable for at least 6 months at room temperature, and the dry solid, upon addition of water, forms a colloidal dispersion having an average particle size less than 200 nm in diameter.

3. A composition as in claim 1, wherein the alpha-tocopherol succinate, analogue or salt thereof is an alpha-tocopheryl hemiester of a short-chain dicarboxylic acidselected from the group consisting of oxalic acid, malonic acid, succinic acid, glutaric acid, adipic acid, pimelic acid, suberic acid, and azelaic acid or a salt of the short-chain dicarboxylic acid.

4. The composition according to claim 1, further comprising an anticancer agent.

5. The composition according to claim 1, wherein the oil component is a triglyceride, diglyceride, or mono-glyceride of a long chain (C14-C22), medium chain (C8-12) or short chain (C4-C6) fatty acid, or a mixture thereof.

6. The composition according claim 1, wherein the oil component is a naturally occurring vegetable oil or animal fat selected from the group consisting of soybean oil, corn oil, sesame oil, coconut oil, safflower oil, cottonseed oil, peanut oil, olive oil, rapeseed oil, palm oil, cholesterol, and mixtures thereof.

7. The composition according to claim 1, wherein the phospholipid is a naturally occurring phospholipid or a synthetic phospholipid.

8. The composition according to claim 7, wherein the naturally occurring phospholipid is selected from the group consisting of soy lecithin, egg lecithin, hydrogenated soy lecithin, hydrogenated egg lecithin, sphingosine, gangliosides, phytosphingosine, and mixtures thereof.

9. The composition according to claim 7, wherein the synthetic phospholipid is selected from the group consisting of diacylglycerols, phosphatidic acids, phosphocholines, phosphoethanolamines, phosphoglycerols, phosphoserines, mixed chain phospholipids, lysophospholipids, pegylated phospholipids, and mixtures thereof.

10. The composition according to claim 1, wherein the antioxidant is selected from the group consisting of edetic acid (EDTA) or salts thereof, ascorbic acid or salts thereof, ascorbyl palmitate, sodium metabisulfite, propyl gallate, butylated hydroxyanisole, butylated hydroxytoluene, tocopherol, reducing sugars, amino acids or salts thereof, citric acid or salts thereof, and mixtures thereof.

11. The composition according to claim 1, wherein the colloidal dispersion is prepared by high shear mixing, high-pressure extrusion, or microfluidization.

12. The composition according to claim 2, wherein the dry solid is prepared by vacuum drying, spray drying or freeze-drying of a composition, comprising alpha-tocopheryl succinate an analogue or salt thereof, at least one component selected from the group consisting of an oil component, phospholipid, and antioxidant: and water; wherein the composition is a colloidal dispersion having an average particle size less than 200 nm in diameter, the alpha-tocopheryl succinate, the analogue or salt thereof is stable for at least 6 months at room temperature and the alpha-tocopheryl succinate has the chemical structure: 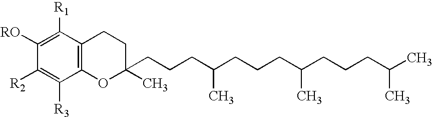
wherein R₁ is CH₃, R₂ is CH₃, R₃ is CH₃, and R is —OOC—(CH₂)₂—COOH.

13. The composition as in claim 2, wherein the cryoprotectant is selected from the group consisting of monosaccharides, disaccharides, polysaccharides, propylene glycol, polyethylene glycol, glycerol, poly-ol, dextrin, cyclodextrin, starch, cellulose and cellulose derivatives, proteins, peptides, amino acids, polyvinypyrrolidone, sodium chloride, and mixtures thereof.

14. The composition according to claim 1, wherein the colloidal dispersion comprises about 1% to about 20% by weight alpha-tocopheryl succinate, an analogue or salt thereof; about 1% to about 20% by weight an oil component; and optionally 0.005%-0.1% by weigh edetic acid sodium salt in an aqueous medium having a pH at between about 6 and about 8; and optionally an osmotic pressure modifier; wherein the colloidal dispersion has an average particle diameter less than about 200 nm.

15. The composition according to claim 2, wherein the dry solid comprises about 1% to 30% by weight alpha-tocopheryl succinate, an analogue or salt thereof; about 1% to about 20% by weight an oil component; about 10% to about 80% by weight cryoprotectant; and optionally about 0.005% to about 1% by weigh edetic acid sodium salt; wherein the dry solid, upon mixing with an aqueous medium, forms a colloidal dispersion having an average particle diameter less than about 200 nm, and a pH at between about 6 and about 8.

16. The composition according to claim 4, wherein the anticancer agent is selected from the group consisting of alkylating agents, antimetabolites, taxanes, cytotoxics, cytoprotectant adjuvants, LHRH analogues, platinum agents, anti-estrogens, anti-androgens, hormones, aromatase inhibitors, cell cycle controlling agents, apoptosis agents, topoisomerase inhibitors, angiogenesis inhibitors, immunotherapy agents, monoclonal antibodies, retinoid, kinase inhibitors and signal transduction inhibitors.

17. The composition according to claim 4, wherein the anticancer agent is paclitaxel or docetaxel.

18. The composition according to claim 4, wherein alpha-tocopheryl succinate, the analogue or salt thereof combined with the anticancer agent produces additive or synergistic anticancer activities.

19. The composition according to claim 1 wherein the loss of intact alpha-tocopheryl succinate, the analogue or salt thereof is no more than about 15% during storage for 6 months at room temperature.

20. The composition according to claim 1 wherein the average particle size does not increase by more than about 50% during storage for 6 months at room temperature.

21. The composition according to claim 2, wherein the dry solid comprises about 1-15% by weight alpha-tocopheryl succinate, about 15-35% by weight lecithin, about 1-5% by weight cholesterol, and about 30-60% by weight cryoprotectant; wherein the dry solid, upon mixing with an aqueous medium, forms a colloidal dispersion having an average particle diameter less than about 200 nm and a pH at between about 6 and about 8.

22. The composition according to claim 1, wherein the colloidal dispersion is a solid-in-water dispersion and comprises about 1-5% by weight alpha-tocopheryl succinate, about 6-10% by weight lecithin, about 0.5-2% by weight cholesterol, and about 10-20% by weight cryoprotectant; wherein the colloidal dispersion having an average particle diameter less than about 200 nm and a pH at between about 6 and about 8.

23. The composition according to claim 2, wherein the dry solid comprises about 1-15% by weight alpha-tocopheryl succinate, about 15-35% by weight lecithin, about 1-5% by weight cholesterol, and about 30-60% by weight cryoprotectant and 0.5-2% by weight paclitaxel or docetaxel; wherein the dry solid, upon mixing with an aqueous medium, forms a colloidal dispersion having an average particle diameter less than about 200 nm and a pH at between about 6 and about 8.

24. The composition of claim 1 wherein the colloidal dispersion is an oil-in-water emulsion or a solid-in-water suspension.

25. The composition of claim 2 wherein the dry solid is an oil-in-solid dispersion or a solid-in-solid dispersion.

26. A method of treating a susceptible neoplasm comprising administering a pharmaceutically effective amount of the composition according to claim 1 to a mammal in need thereof.

27. The method according to claim 26 wherein the administration is by an injection route selected from the group consisting of intravenous, intraabdominal, intraarterial, intraarticular, intracapsular, intracervical, intracranial, intraductal, intradural, intralesional, intralocular, intralumbar, intramural, intraocular, intraoperative, intraparietal, intraperitoneal, intrapleural, intrapulmonary, intraspinal, intrathoracic, intratrachcal, intratympanic, intrauterine, and intraventricular administration.

28. The method according to claim 26, wherein the mammal is human.

29. The method of claim 26, wherein the susceptible neoplasm is selected from the group consisting of leukemias, sarcomas, carcinomas, and myelomas.

30. The method of claim 26 wherein the composition according to claim 1 or claim 2 is administering to the mammal in need thereof intravenously.
